(19) United States
(12) Patent Application Publication
(10) Pub. No.: US 2025/0205508 A1
(43) Pub. Date: Jun. 26, 2025

(54) A FACE MASK FOR PROVIDING A THERAPY TO A USER

(71) Applicant: Shenzhen Kaiyan Medical Equipment Co., Ltd., Shenzhen (CN)

(72) Inventors: Alain Dijkstra, Amstelveen (NL); LI XIANG, Shenzhen (CN); Dong Jianheng, Shenzhen (CN)

(73) Assignee: Shenzhen Kaiyan Medical Equipment Co., Ltd., Shenzhen (CN)

(21) Appl. No.: 18/973,142

(22) Filed: Dec. 9, 2024

(30) Foreign Application Priority Data
Dec. 26, 2023 (CN) 202311806032.6
Dec. 26, 2023 (CN) 202323559486.6

(51) Int. Cl. A61N 5/06

(52) U.S. Cl. CPC A61N 5/06; A61N 2005/0626; A61N 2005/0632; A61N 2005/0647; A61N 2005/0652

(57) ABSTRACT

Embodiments of the present invention provide a face mask for providing a therapy to a user. The face mask comprises a mask body and a control box. One end of the control box is connected with the mask body through a connecting band. The mask body comprises an outer layer, an inner layer and a circuit board positioned between the outer layer and the inner layer. The circuit board is provided with a plurality of LEDs for providing phototherapy. The mask body is provided with a slot to tighten a strap. The strap is provided with a connecting piece at the center to which the control box is configured to detachably connect.

### TECHNICAL FIELD

The present invention generally relates to a therapeutic device and, more particularly, to a face mask to provide therapeutic effects to a user.

### BACKGROUND ART

A phototherapy mask is a cosmetic device that uses an LED light source for facial skin care. The phototherapy mask offers benefits, such as improving skin texture, improving blood circulation, boosting immunity, and reducing skin concerns such as wrinkles and dark spots. Phototherapy masks use LED light sources of different wavelengths, including red, blue, yellow, and green, to select the appropriate light therapy mode for individual skin types. Phototherapy masks are easy to use, and can be used by anyone at home without the need for professional skills or equipment.

With varied treatment regimes, the phototherapy mask need to store information and regulate the functioning of LED light source in multiple ways. To accomplish this, the phototherapy device comes equipped with a controller that stores the instructions for different treatment regime and regulate the functioning of LED light source. The controller also facilitates controlling the phototherapy mask by an external communication device.

The conventional phototherapy mask generally comprises a mask body, a controller and a connecting wire. The mask body comprises a mask silicone layer, the lower side of the mask silicone layer is provided with a masking sheet, a soft PCB lamp board is installed on the lower side of the masking sheet. A type-C charging interface small board is arranged at the upper front end position of the masking sheet, a surface cloth is arranged on the lower side of the flexible PCB light board.

In the existing phototherapy mask, the controller needs to be handheld while performing the phototherapy, and if the controller is added to the mask, it will increase the weight of the mask and cause it to fall off easily.

In order to overcome the disadvantage associated with the prior art, the present invention provides a face mask with a controller positioned in an ergonomic manner and that can be worn comfortably by a user.

### OBJECTS OF THE INVENTION

Some of the objects of the invention are as follows:

An object of the present invention is to provide a face mask that is stable to wear and easy to use.

Another object of the present invention is to provide an ergonomic design of a phototherapy face mask with controller attached with the face mask.

Another object of the present invention is to provide a face mask with a controller that can be positioned relative to the face mask without changing the center of gravity and avoid falling off the mask.

Another object of the present invention is to provide a face mask with a controller that can be worn by a user comfortably while the user is moving or when the user is lying down.

Another object of the present invention is to provide a face mask that provide phototherapy as well as other alternate therapy to the user

### SUMMARY OF THE INVENTION

According to a first aspect of the present invention, a face mask for providing a therapy to face of a user is provided. The face mask comprising: an outer layer; an inner layer; a circuit board positioned between the outer layer and the inner layer; a plurality of LEDs arranged on the circuit board; a pair of slot in the face mask to install a strap to secure the face mask around a user's head; a connecting band originating from a forehead portion of the face mask having a connecting piece to attach with the strap at the back of the user's head; a control box affixed to the connecting band.

In one embodiment of the invention, the control box is electrically connected to the circuit board.

In one embodiment of the invention, a first end of the connecting band is attached to the forehead portion of the face mask and a second end of the connecting band is connected to the control box such that the connecting band extends backward along the direction of forehead of the user to connect with the strap.

In one embodiment of the invention, the second end of the connecting band is provided with a plug and the control box has a jack to connect the connecting band with the control box.

In one embodiment of the invention, the control box comprises a positioning plate fixably connected with the connecting band, wherein the positioning plate is provided with a battery, a set of control buttons, a control circuit board electrically connected with the battery and a control box cover.

In one embodiment of the invention, the control circuit board is configured to connect with an external communication device to receive command and instructions for controlling the face mask.

In one embodiment of the invention, the outer layer comprises a fixed column and the circuit board comprises a first fixed hole such that the fixed column passed through the first fixed hole.

In one embodiment of the invention, the circuit board is provided with an opaque masking layer comprising a light bead hole and a second fixed hole, such that the plurality of LEDs passes through the light bead hole and the fixed column passes through the second fixed hole.

In one embodiment of the invention, the face mask comprises an eye opening detachably connected with an eye protective cover.

In one embodiment of the invention, the connecting band further comprises a plurality of LEDs along the length of the connecting band.

In one embodiment of the invention, the control box is positioned at the back of the user's head when the face mask is worn by the user.

According to a second aspect of the present invention, a face mask for providing a therapy to face of a user is provided. The face mask comprising: an outer layer; an inner layer; a circuit board positioned between the outer layer and the inner layer; a plurality of LEDs arranged on the circuit board; a pair of slot in the face mask to install a strap to secure the face mask around a user's head; a connecting band originating from a forehead portion of the face mask having a connecting piece to attach with the strap at the back of the user's head; a control box affixed to the connecting band or the strap.

In one embodiment of the invention, the face mask further comprising at least one stimulation element selected from a group consisting of a heating element, a cooling element, a peltier element, a micro-current electrode.

In one embodiment of the invention, a first end of the connecting band is connected to the forehead portion of the face mask and a second end of the connecting band is connected to the control box such that the connecting band extends backward along the direction of forehead of the user to connect with the connecting piece on the strap.

In one embodiment of the invention, the control box comprises a positioning plate fixably connected with the connecting band, wherein the positioning plate is provided with a battery, a control circuit board electrically connected with the battery and a control box cover.

In one embodiment of the invention, the control circuit board is configured to connect with an external communication device to receive command and instruction for controlling the face mask.

In one embodiment of the invention, the connecting band further comprises a plurality of LEDs along the length of the connecting band.

In one embodiment of the invention, the control box is positioned at the back of the user's head when the face mask is worn by the user.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of the specification, the phrase “communication network” refers to a group of several connected devices including computing devices (such as desktops, mobile handheld devices, tablet PCs, notebooks, etc.), local and remotely located servers (such as web servers, application servers, database servers, Application Program Interface (API) servers, load balancers, compute nodes, and the like), routers, antennas, modems, multiplexers, demultiplexers, and the like. In that regard, the aforementioned connected devices may be able to exchange data signals through wired and/or wireless means as per several combinations of several different communication protocols such as 802.11 (Wi-Fi), 802.3 (Ethernet), Bluetooth, NFC, ZigBee and 3GPP protocols such as HSPA, HSDPA, LTE, GSM, CDMA, WLL and the like.

In the context of the specification, when an element is referred to as being “fixed to” or “disposed to” another element, it may either directly on another element or indirectly on that other element. When a component is said to be “connected” or “connected to” another component, it may be directly connected to another component or indirectly connected to other component on the piece.

In the context of the specification, the terms “first’, “second” and “third” are only used for descriptive purpose and does not implicate the relative importance or to implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to Nth Degree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 shows a perspective view of a phototherapy face mask with a controller in accordance with an embodiment of the present invention.

FIG. 2 shows an exploded view of the phototherapy face mask in accordance with an embodiment of the present invention.

FIG. 3 shows a front view of the phototherapy face mask in accordance with an embodiment of the present invention.

FIG. 4 shows the phototherapy face mask with the controller worn by a user in accordance with an embodiment of the present invention.

FIG. 5 shows a phototherapy face mask with connecting band originating from the cheek portion and running parallel to the strap in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a face mask for providing therapeutic treatment to a user. The face mask comprises a mask body and a control box connected with the mask body through a connecting band. The mask body comprises an outer layer and an inner layer. On one side of the circuit board, a plurality of LEDs are arranged on the circuit board. The mask body is provided with a pair of slot on the opposite side near the cheek area for fastening a strap. The strap is provided with a connecting piece in middle such that the strap when fastened, the connecting piece is positioned at back of the head. The control box is detachably connected with the strap through the connecting piece. The connecting band connects the mask body with the control box and the control box is connected and fixed with the strap through the connecting piece. This enhances the stability of the mask body wearing and it is convenient to use.

Several additional functionalities may also be added to the therapeutic device. For example, the mask body is provided with a medicament storage chamber and a plurality of fluid channels to dispense the medicament over the face of the user. The face mask comprises one or more stimulation element, such as heating element, cooling element, massage head, micro-current element to provide one or more stimulation therapy. The control box in the face mask is in communication with an external communication device The external communication device can send commands and instruction to the control box or regulating the functioning of one or more stimulation element.

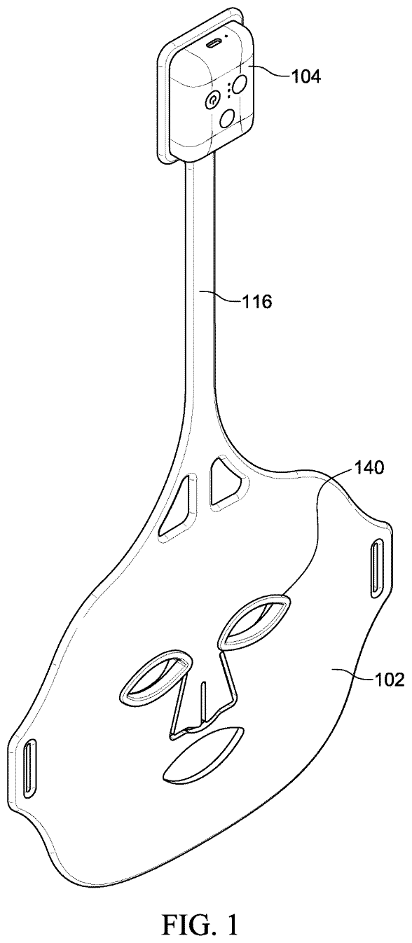

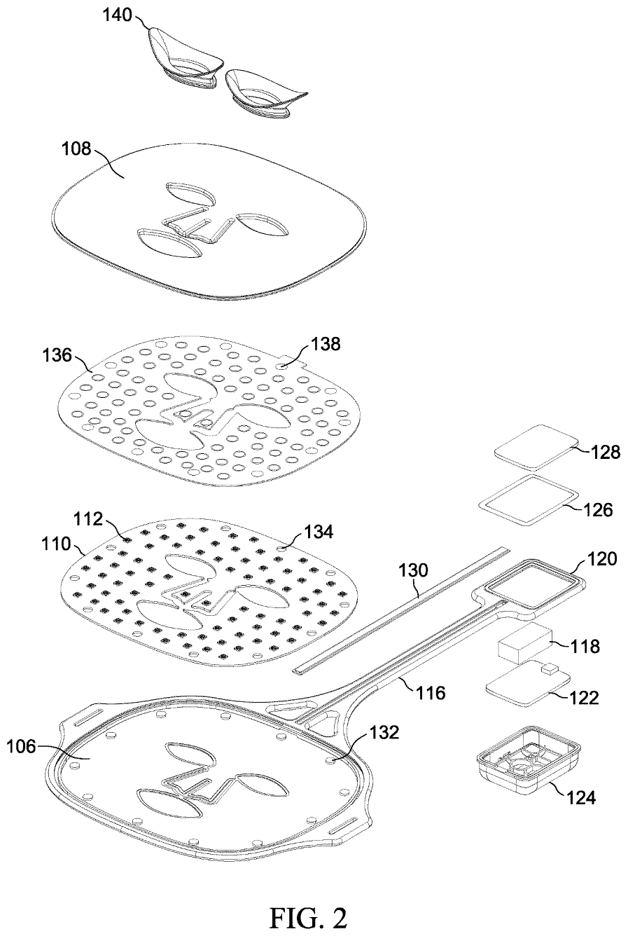

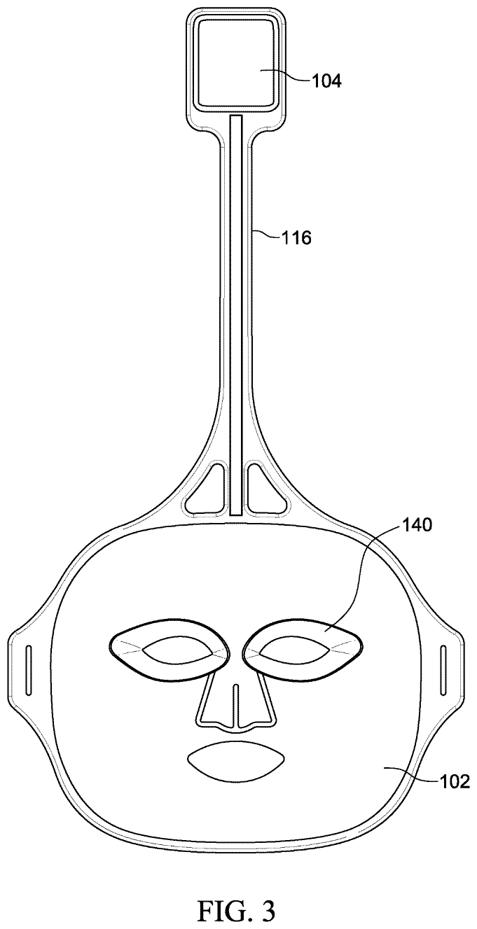

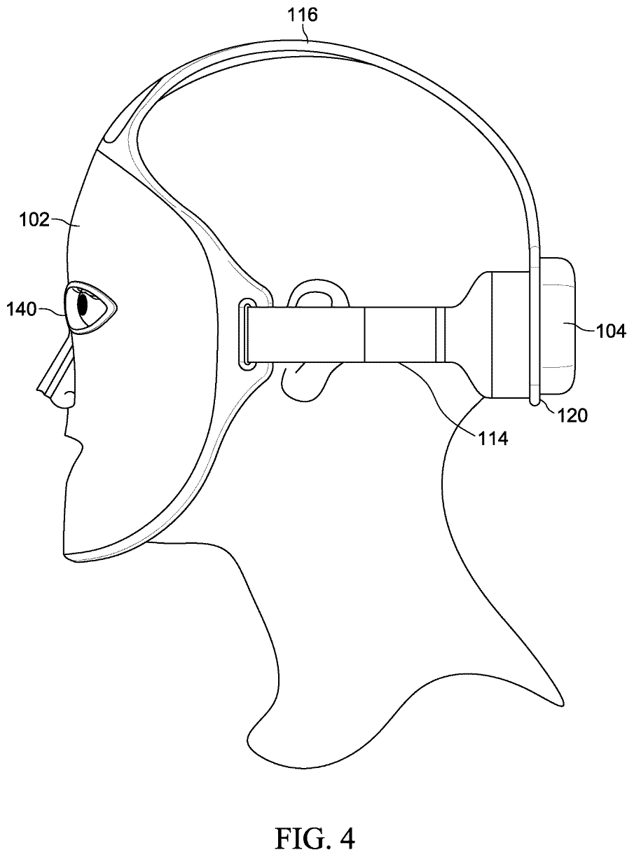

Referring to FIG. 1 to FIG. 4, a face mark to provide therapy to a user is shown. The face mask comprises a mask body 102 and a control box 104. One end of the control box 104 is connected with the mask body 102 through a connecting band 116. The mask body 102 comprises an outer layer 106, an inner layer 108 and a circuit board 110 positioned between the outer layer 106 and the inner layer 108. A plurality of LEDs 112 are arranged on one side of the circuit board 110 towards the inner layer 108. The mask body 102 is provided with a pair of slots, each of the slot is respectively arranged at the edges of two sides of the cheeks of the mask for installing a strap 114. The strap 114 is located on both sides of the face of the mask body for fixing the mask body with the human head. The strap 114 is provided with a connecting piece at the middle position that winds around the rear of the head of the user.

The mask body 102 comprises a forehead portion and a cheek portion. The connecting band 116 is connected to the forehead portion. The connecting band 116 extends backward along the human forehead, and the control box 104 at the other end connects with the connecting piece on the strap 114. The length of the connecting band 116 can make the control box 104 be able to be detachably connected with the strap 114 through the connecting piece. The detachable connection between the control box 104 and the strap 114 eliminates the need of the user to hold the control box 104 when using the face mask for phototherapy or other therapy, and can walk freely while the mask is ON. When the user is lying down and uses the face mask, the control box 104 can be disassembled with the strap 114, and the control box is placed aside to increase the comfort of use. The connecting band 116 is arranged on the mask body 102 close to the forehead part of the human body, so that the mask body 102 can be worn and fixed without being easy to fall off.

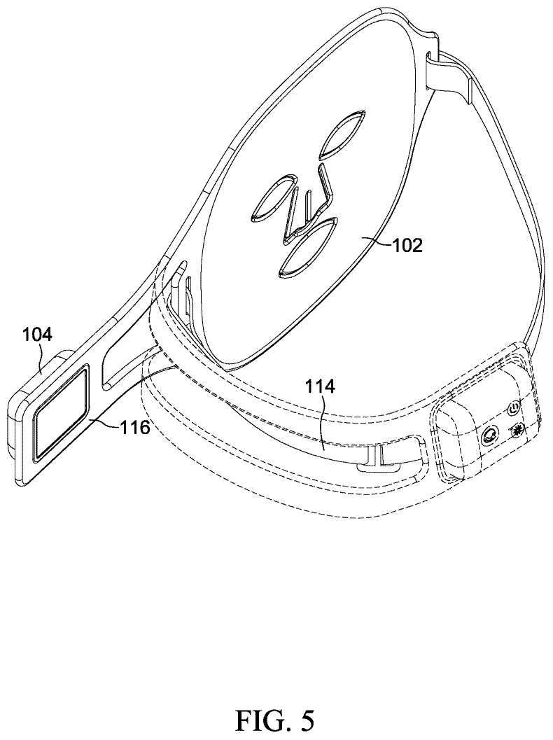

In an embodiment, the connecting band 116 can also be arranged in other positions, such as being arranged parallel to the strap 114 on both sides of the mask body, and the control box 104 can still be fixed on the band or removed. Alternatively, the control box is positioned on the strap 114 and the connecting band connects with the strap 114. FIG. 5 shows the face mask with horizontal connecting band. The face mask 102 is provided with a pair of slots on cheek portion for attaching the strap 114. The connecting band 116 originate parallel to the strap 114 and extends halfway towards the back of a user's head. The control box 104 is located at the end of the connecting band 116. The connecting band 116 runs parallel to the strap 114 and the control box 104 gets attached with the connecting piece at the center of the strap.

The control box 104 is provided with a battery 118. The battery 118 is electrically connected with the circuit board 110 to supply power to the plurality of LEDs 112 or other electrical component. The control box 104 comprises a positioning plate 120, and the positioning plate 120 is connected and fixed with the connecting band 116. One side of the positioning plate 120 is provided with the battery 118 and a control circuit board 122. The control circuit board 122 is electrically connected with the battery 118. The battery 118 and the control circuit board 122 are provided with a control box cover 124. The control box 104 has a set of control buttons to control the functioning of the plurality of LEDs 112. The positioning plate 120, connecting band 116 and the outer layer 106 are integrally formed as a single structure unit. The other side of the positioning plate 120 is provided with a seal 126 and control box bottom cover 128. The control box bottom cover 128 can be coupled with the control box outer cover. Because the positioning plate 120 is integrally formed with the connecting band 116 and the outer layer 106, the positioning plate 120 is made of flexible material, and the control box bottom cover 128 can be made of a hard solid material.

The connecting band 116 comprises a sealing band 130, and the connecting band 116 is provided with a long opening. The sealing band 130 is arranged in the long opening, so that the control box 104 is electrically connected with the circuit board 110 using conductive wire.

In an embodiment of the present invention, the connecting band 116 is also provided with a plurality of LEDs along the length of the connecting band 116. The connecting band covers the top portion of the human head, therefore can be used to provide phototherapy on the top portion of the head.

The outer layer 106 is provided with a fixed column 132, and the circuit board 110 is provided with a first fixed hole 134. The fixed column 132 passes through the first fixed hole 134 to fix the circuit board. The circuit board 110 is further provided with an opaque masking layer 136. The opaque masking layer 136 is provided with a light bead hole and a second fixed hole 138. The plurality of LEDs 112 passes through the light bead hole, and the fixed column 132 passes through the second fixed hole 138.

The mask body 102 is flexible, and the inner layer 108 is a light-transmitting layer. The mask body 102 has an eye opening, and an eye shield 140 is detachably connected at the eye opening.

In an embodiment, the connecting piece is a Velcro or a magnetic suction piece or a buckle that is fixed on the control box 104, and the strap 114 has a corresponding Velcro or a magnetic part or a buckle.

In an embodiment, the connecting band 116 is provided with a plug to connect with the control box. The control box 104 is provided with a jack corresponding to the plug. The control box 104 is provided with a charging port, which is electrically connected with the control circuit board 122 to charge the battery 118. The control box 104 can be charged after disassembling the connecting band 116. When the battery 118 is charging, the plug can be connected with an external power supply to power the mask body and the mask can be used without any interference.

In an embodiment of the present invention, the face mask also has a micro-current element present between the outer layer 106 and the inner layer 108. The micro-current element is electrically connected with the circuit board 110. The micro-current element comprises an electrode contact that stimulate the facial skin of the user using a micro-current.

In another embodiment of the present invention, the face mask further comprises a medicament storage chamber that stores a liquid medicament. A plurality of pores are provided in the inner layer 108 that are connected with the medicament storage chamber. A flexible button is provided on the top or front of the face mask. When the user presses the flexile button, the button squeezes the medicament storage chamber to dispense the liquid medicament from the plurality of pores. The liquid medicament is dispensed around the face of the user. When used with micro-current therapy, the liquid medicament augment the therapy effect. The face mask comprises a plurality of fluid channels that circulates the liquid medicament from the medicament storage chamber to the plurality of pores.

In an embodiment of the present invention, the face mask of the present invention is provided with one or more stimulation elements, such as a heating element or a cooling element. When the heating element is used, the heating element provide heat therapy to the user. When the cooling element is used, the cooling element provide cooling effect to the user.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A face mask for providing therapy to face of a user, the face mask comprising:
an outer layer;
an inner layer;
a circuit board positioned between the outer layer and the inner layer;
a plurality of LEDs arranged on the circuit board;
a pair of slot in the face mask to install a strap to secure the face mask around a user's head;
a connecting band originating from a forehead portion of the face mask having a connecting piece to attach with the strap at back of the user's head; and
a control box affixed to the connecting band.

2. The face mask of claim 1, wherein the control box is electrically connected to the circuit board.

3. The face mask of claim 1, wherein a first end of the connecting band is attached to the forehead portion of the face mask and a second end of the connecting band is connected to the control box such that the connecting band extends backward along the direction of forehead of the user to connect with the strap.

4. The face mask of claim 3, wherein the second end of the connecting band is provided with a plug and the control box has a jack to connect the connecting band with the control box.

5. The face mask of claim 1, wherein the control box comprises a positioning plate fixably connected with the connecting band, wherein the positioning plate is provided with a battery, a set of control buttons, a control circuit board electrically connected with the battery and a control box cover.

6. The face mask of claim 5, wherein the control circuit board is configured to connect with an external communication device to receive command and instruction for controlling the face mask.

7. The face mask of claim 1, wherein the outer layer comprises a fixed column and the circuit board comprises a first fixed hole such that the fixed column passed through the first fixed hole.

8. The face mask of claim 7, wherein the circuit board is provided with an opaque masking layer comprising a light bead hole and a second fixed hole, such that the plurality of LEDs passes through the light bead hole and the fixed column passes through the second fixed hole.

9. The face mask of claim 1, wherein face mask comprises an eye opening detachably connected with an eye protective cover.

10. The face mask of claim 1, wherein the connecting band further comprises a plurality of LEDs along the length of the connecting band.

11. The face mask of claim 1, wherein the control box is positioned at the back of the user's head when the face mask is worn by the user.

12. A face mask for providing therapy to face of a user, the face mask comprising:
an outer layer;
an inner layer;
a circuit board positioned between the outer layer and the inner layer;
a plurality of LEDs arranged on the circuit board;
a pair of slot in the face mask to install a strap to secure the face mask around a user's head;
a connecting band originating from a forehead portion of the face mask having a connecting piece to attach with the strap at back of the user's head; and
a control box affixed to the connecting band or the strap.

13. The face mask of claim 12, wherein the face mask further comprising at least one stimulation element selected from a group consisting of a heating element, a cooling element, a peltier element, a micro-current electrode.

14. The face mask of claim 12, wherein a first end of the connecting band is connected to the forehead portion of the face mask and a second end of the connecting band is connected to the control box such that the connecting band extends backward along the direction of forehead of the user to connect with the connecting piece on the strap.

15. The face mask of claim 12, wherein the control box comprises a positioning plate fixably connected with the connecting band, wherein the positioning plate is provided with a battery, a control circuit board electrically connected with the battery and a control box cover.

16. The face mask of claim 15, wherein the control circuit board is configured to connect with an external communication device to receive command and instruction for controlling the face mask.

17. The face mask of claim 12, wherein the connecting band further comprises a plurality of LEDs along the length of the connecting band.

18. The face mask of claim 12, wherein the control box is positioned at the back of the user's head when the face mask is worn by the user.

19. A face mask for providing therapy to face of a user, the face mask comprising:
an outer layer;
an inner layer;
a circuit board positioned between the outer layer and the inner layer;
a plurality of LEDs arranged on the circuit board;
a pair of slot in the face mask to install a strap to secure the face mask around a user's head;
a connecting band originating from the pair of slots of the face mask that runs parallel to the strap, the strap having a connecting piece at back of the user's head; and
a control box affixed to the connecting band; and
wherein the control box on the connecting band attached with the connecting piece on the strap at the back of the user's head.
